(19) United States
(12) Patent Application Publication
(10) Pub. No.: US 2014/0371179 A1
(43) Pub. Date: Dec. 18, 2014

(54) Methods and Compositions for Treating Esophageal Diseases

(71) Applicant: Chris V. Simmons, Sugar Land, TX (US)

(72) Inventor: Chris V. Simmons, Sugar Land, TX (US)

(73) Assignee: PROFESSIONAL COMPOUNDING CENTERS OF AMERICA, Houston, TX (US)

(21) Appl. No.: 13/917,275

(22) Filed: Jun. 13, 2013

(51) Int. Cl. A61K 31/58; A61K 45/06

(52) U.S. Cl. CPC A61K 31/58; A61K 45/06
USPC 514/170; 514/174; 514/171

(57) ABSTRACT

Compositions and methods for treating esophageal diseases are disclosed. More specifically, these methods may refer to the treament of esophageal varices and eosinophilic esophagitis using an oral suspension drug for humans. The oral suspension drug may include poloxamer f127 used as vehicle and budesonide as a corticosteroid. Poloxamer oral suspension may be administrated by swallowing the poloxamer oral suspension drug which may slide down through the esophageal and may be adhered in the affected site providing a longer residence time. Additionally, poloxamer oral suspension may be mixed with APIs such as antifungals, antibacterials, and corticosteroids, previously dissolved in a suitable liquid, such as water.

### CROSS-REFERENCE TO RELATED APPLICATIONS

N/A

### BACKGROUND

1. Field of the Disclosure

The present disclosure relates generally to esophageal diseases, and more particularly, to compositions and methods for treating eosinophilic esophagitis and esophageal varices.

2. Background Information

Incidence of gastrointestinal functional diseases increases day by day, where the common esophageal diseases can be reflux esophagitis, esophageal cancer, esophageal stenosis, esophageal varices, dyspepsia, and functional dysphagia. In the course of medical diagnosis and treatment, a continuous monitoring or treatment of the esophagus is often required. Currently, there are many types of esophagus treatments on the market such as endoscopic therapy, surgery, injection and oral suspension drugs, among others.

Oral suspension drugs may commonly include active pharmaceutical ingredients (APIs) for treating esophageal diseases. For example, esophageal varices is a common disease in humans that may be treated with budesonide oral suspension with vehicles, such as methylcellulose, however, methylcellulose, used as thickener within budesonide oral suspension does not have bio-adhesive properties.

Current oral suspensions for treating esophageal diseases generally slide down without adhering to the affected site. Therefore, there is a need for suitable pharmaceutical oral compositions that include a vehicle which allows APIs remain or adhere (for a suitable period of time) on the affected esophageal site and may allow a faster healing time.

### SUMMARY

According to various embodiments, the present disclosure relates to compositions and methods for the treatment of eosinophilic esophagitis and esophageal varices in humans. More specifically, the present disclosure refers to the application of an oral pharmaceutical composition in the mouth which may enable an effective administration, thus improving treatment outcomes. The oral composition may include about 5% to 10% of poloxamer f127 (as a vehicle); with budesonide in an amount of about 1 to 2 ring/10 ml. According to another embodiment poloxamer f127 may have the ability to solubilize in soluble drugs and poloxamer f127 may exhibit an effective and stable vehicle for oral compositions.

In another embodiment, oral composition may include a second and/or third API in combination with budesonide. In another embodiment, oral composition may include API or APIs other than budesonide. Poloxamer f127 may be used with any suitable API such as antibacterials, antifungals, corticosteroids, antiparasitics, among others. According to one embodiment, the poloxamer oral suspension may start to thicken when it is placed in the mouth because of the thermoreversible properties. Poloxamer oral suspension may be administrated once a day in the morning.

According to some embodiments, the poloxamer oral suspension may have bio-adhesive properties and may increase the micro circulation which may allow healing process of the body immune system. Consequently, when poloxamer oral suspension is swallowed, poloxamer oral suspension may slide down through the pharynx into the esophagus and may travel via peristalsis to the affected site in the esophagus where poloxamer oral suspension may be adhered, providing a longer residence time.

Numerous other aspects, features and benefits of the present disclosure may be made apparent from the following detailed description taken together with the drawing figures.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 is an oral suspension components block diagram, according to an embodiment.

FIG. 2 depicts a close up view of lower esophageal, where poloxamer oral suspension may be used in esophageal varices, according to an embodiment.

FIG. 3 depicts a close up view of lower esophageal, where poloxamer oral suspension may be used in eosinophilic esophagitis, according to an embodiment.

### DETAILED DESCRIPTION

The present disclosure is here described in detail with reference to embodiments illustrated in the drawings, which form a part hereof. Other embodiments may be used and/or other changes may be made without departing from the spirit or scope of the present disclosure. The illustrative embodiments described in the detailed description are not meant to be limiting of the subject matter presented here.

### Definitions

As used here, the following terms may have the following definitions:

“Treating” and “treatment” refers to a reduction in severity and/or frequency of symptoms, elimination of symptoms and/or underlying cause, prevention of the occurrence of symptoms and/or their underlying cause, and improvement or remediation of damage.

“Poloxamer f127” refers to a non-toxic, di-block copolymer of polyoxyethylene and polyoxypropylene where in aqueous solutions (30% w/v) show thermoreversible gelation, being liquid at temperatures <15 degrees C. and robust gels at temperatures >15 degrees C.

“Budesonide” refers to a class of medications called corticosteroids that may work by decreasing inflammation (swelling) in the digestive tract of people or animals.

“Esophageal varices” refers to abnormal enlarged veins in the lower part of the esophagus and may occur most often in people with serious liver diseases.

“Eosinophilic esophagitis” refers to an allergic reaction that causes inflammation and damage to the esophagus which may be usually caused by a food allergy.

“Active pharmaceutical ingredient” refers to a chemical material or compound that induces a desired pharmacological, physiological effect, and includes agents that are therapeutically effective, prophylactically effective, or cosmeceutical effective.

“vehicles” refer to carrier materials suitable for transdermal/topical or oral drug administration.

### Description of the Drawings

### Oral Suspension Composition

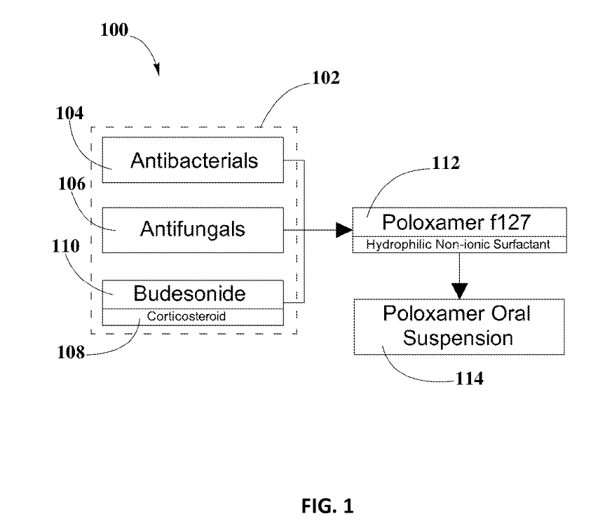

FIG. 1 is an oral suspension components block diagram 100. According to some embodiments, APIs 102 may include the active ingredients that may be used for treating the esophageal diseases. APIs 102 may include antibacterials 104, antifungals 106, and corticosteroids 108. Suitable corticosteroid 108 may be budesonide 110 compositions that may include about 1 to 2 ring/10 ml of budesonide 110.

According to one embodiment, APIs 102 may be combined with a vehicle such as poloxamer f127 112. Suitable concentration of poloxamer f127 112 for the disclosed oral suspension may be of about 5% to about 10%. Poloxamer f127 112 may have thermoreversible properties, for instance, poloxamer f127 112 at room temperature is in liquid state changing to a gel at body (or warm) temperature.

According to one embodiment, APIs 102 may be mixed with poloxamer f127 112, previously dissolved in a suitable solvent, resulting in poloxamer oral suspension 114 which may be effective for treating esophageal varices, eosinophilic esophagitis, among other esophageal diseases. Additionally, APIs 102 may be mixed with poloxamer f127 112 for treating other esophageal disorders, such as structural abnormalities, motility abnormalities, inflammatory disorders, and malignancies. In one embodiment, suitable solvent to dissolve poloxamer f127 112 may be water, other embodiments may include other suitable solvents.

Other embodiments may include other suitable additives such as pH adjusting agents, preservatives, emulsifiers, opacifiers, antioxidants, fragrances, colorant, gelling agents, thickening agents, stabilizers, surfactants, among others. Other agents may also be added, such as antimicrobial agents, to prevent spoilage upon storage, for example, to inhibit growth of microbes such as yeasts and molds. Suitable antimicrobial agents are typically selected from the group consisting of the methyl and propyl esters of p-hydroxybenzoic acid, sodium benzoate, sorbic acid, imidurea and combinations thereof.

### Active Pharmaceutical Ingredients

Poloxamer f127 may be used with any suitable API such as antibacterials, antifungals, corticosteroids, antiparasitics, among others.

Antiviral Agents

According to some embodiments, antiviral agents may include acyclovir, famciclovir and valacyclovir. Other antiviral agents include abacavir, aciclovir, adefovir, amantadine, amprenavir, arbidol., atazanavir, artipla, brivudine, cidofovir, combivir, edoxudine, efavirenz, emtricitabine, enfuvirtide, entecavir, fomvirsen, fosamprenavir, foscarnet, fosfonet, ganciclovir, gardasil, ibacitabine, immunovir, idoxuridine, imiquimod, indinavir, inosine, integrase inhibitors, interferons, including interferon type Ill, interferon type II, interferon type I, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, tenofovir, tenofovir disoproxil, tipranavir, trifluridine, trizivir, tromantadine, truvada, valganciclovir, vicriviroc, vidarabine, viramidine, zalcitabine, zanamivir, zidovudine, and combinations thereof.

Antibacterial Agents

According to some embodiments antibacterial agents may include amikacin, gentamicin, kanamycin, neomycin, netilmicin, streptomycin, tobramycin, paromomycin, geldanamycin, herbimycin, loracarbef, ertapenem, doripenem, imipenem, cilastatin, meropenem, cefadroxil, cefazolin, cefalotin, cefalexin, cefaclor, cefamandole, cefoxitin, defprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillan, bacitracin, colistin, polymyxin B, ciprofloxacin, enoxacin, gatifloxacin, levofloxacin, lomefloxacin, moxifloxacin, norfloxacin, ofloxacin, trovfloxacin, mafenide, prontosil, sulfacetamide, sulfamethizole, sulfanimilimde, sulfasalazine, sulfisoxazole, trimetoprim, demeclocycline, doxycycline, minocycline, oxytetracycline, tetracycline, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinuspristin/dalfopristin, rifampin, tinidazole, AL-15469A (Alcon Research), AL-38905 (Alcon Research) and combinations thereof.

Antifungal Agents

Accoriding to some embodiments, antifungal agents may include amrolfine, utenafine, naftifine, terbinafine, flucytosine, fluconazole, itraconazole, ketoconazole, posaconazole, ravuconazole, voriconazole, clotrimazole, econazole, miconazole, oxiconazole, sulconazole, terconazole, tioconazole, nikkomycin Z, caspofungin, micafungin, anidulafungin, amphotericin B, liposomal nystastin, pimaricin, griseofulvin, ciclopirox olamine, haloprogin, tolnaftate, undecylenate, clioquinol, and combinations thereof.

Aniparasitic Agents

According to some embodiments, antiparasitic agents may include amitraz, amoscanate, avermectin, carbadox, diethylcarbamizine, dimetridazole, diminazene, ivermectin, macrofilaricide, malathion, mitaban, oxamniquine, permethrin, praziquantel, prantel pamoate, selamectin, sodium stibogluconate, thiabendazole, and combinations thereof.

Corticosteroids

According to some embodiments, corticosteroids may include hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone.

Anaesthetics Agents

According to some embodiments, anaesthetics agents may include benzocaine, butamben picrate, tetracaine, dibucaine, prilocalne, etidocaine, mepivacaine, bupivicaine, and lidocaine. Preferred non-steroidal anti-inflammatory agents may include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine. Preferred steroids may include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone, and combinations thereof.

### Poloxamer Oral Suspension (Applications)

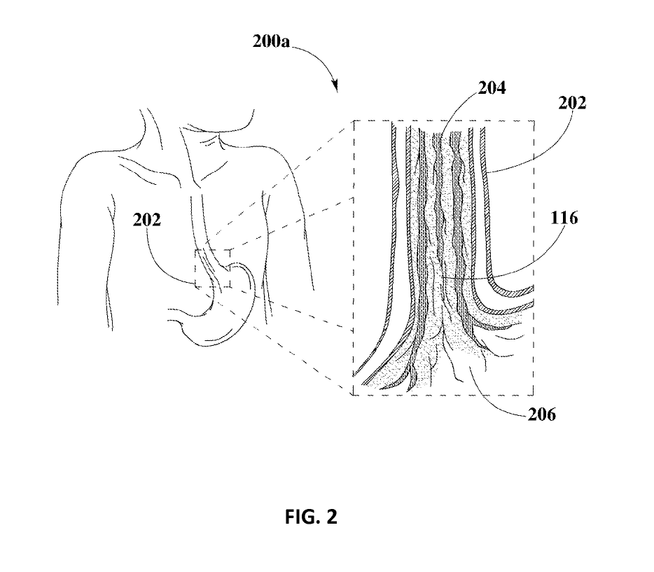

FIG. 2 depicts close up view 200a of lower esophagus 202 where poloxamer oral suspension 116 may be used for treating esophageal varices 204. Specifically, esophageal varices 204 may be abnormal, enlarged veins located in lower esophagus 202 and upper stomach 206. Esophageal varices 204 may occur most often in people with serious liver diseases and may be developed when normal blood flow to the liver is slowed. Consequently, the blood may then back up into nearby smaller blood vessels, such as those in lower esophagus 202, causing the vessels to swell. Sometimes, esophageal varices 204 may rupture causing life-threatening bleeding.

The primary aim for treating esophageal varices 204 is to prevent bleeding. Bleeding esophageal varices 204 are life-threatening. According to some embodiments, poloxamer oral suspension 116 may be used for treating esophageal varices 204 applying a dosage of about 5 ml to about 10 ml once a day. Poloxamer oral suspension 116 may include bio-adhesive properties and may also increase micro-circulation. Poloxamer oral suspension 116 may be adhered to the walls of lower esophagus 202 which may give a longer residence time.

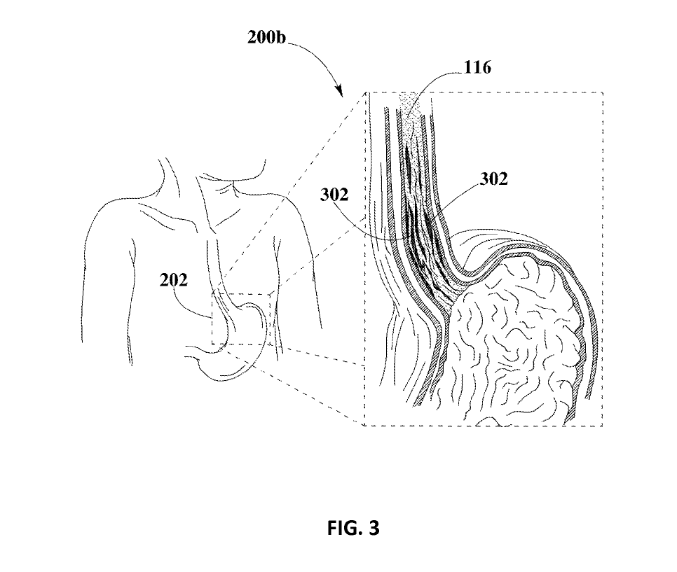

FIG. 3 depicts close up view 200b of lower esophagus 202 where poloxamer oral suspension 116 may be used in eosinophilic esophagitis 302. Specifically, eosinophilic esophagitis 302 may be an inflammation which was primarily attributed to acid reflux esophagitis, but in the last 5 years eosinophilic esophagitis 302 (also known as allergic esophagitis, primary eosinophilic esophagitis, and idiopathic eosinophilic esophagitis) has emerged as an important independent clinicopathologic entity found to affect children and adults. Eosinophilic esophagitis 302 may commonly affect lower esophagus 202

According to some embodiments, eosinophilic esophagitis 302 may be treated using poloxamer oral suspension 116 administrating a dosage of about 5 ml to about 10 ml once a day. Poloxamer oral suspension 116 may be adhered (because of the bio-adhesive properties) at the desired site of action for a longer residence time.

### EXAMPLES

Example #1 is an embodiment of poloxamer oral suspension 116 application, which may be used in esophageal diseases in animals, administrating a dosage of about 0.25 ml to about 20.0 ml (depend on animal weight/size) of poloxamer oral suspension 116 once a day.

While various aspects and embodiments have been disclosed here, other aspects and embodiments may be contemplated. The various aspects and embodiments disclosed here are for purposes of illustration and are not intended to be limiting, with the true scope and spirit being indicated by the following claims.

1. A pharmaceutical composition for oral administration comprising
(a) budesonide in a concentration of about 1 mg/10 ml to about 2 mg/10 ml; and
(b) about 5% to about 10% by weight poloxamer f127;
(c) imidurea; and
(d) optionally, an antimicrobial agent selected from the group consisting of methyl ester of p-hydroxybenzoic acid, propyl ester of p-hydroxybenzoic acid, sodium benzoate, sorbic acid, and combinations thereof.

2. A composition according to claim 1, which is in the form of an oral suspension.

3. A composition according to claim 1, which is in the form of a tablet.

4. A composition according to claim 1, further comprising an antibacterial composition, an antifungal composition, a corticosteroid, an antiparasitic composition, or a combination thereof.

5. A method of treating esophageal disease, comprising orally delivering to a patient an effective amount of a pharmaceutical composition that comprises budesonide and at least one poloxamer.

6-8. (canceled)

9. The method according to claim 5, wherein the pharmaceutical composition is administered once per day.

10. The method according to claim 5, wherein the pharmaceutical composition is administered in multiple doses per day.

11. The method according to claim 5, wherein the pharmaceutical composition is in the form of an oral suspension.

12. The method according to claim 5, wherein the pharmaceutical composition is in the form of a tablet.

13. The method according to claim 5, wherein the pharmaceutical composition further comprising an antibacterial composition, antimicrobial agent , an antifungal composition, a corticosteroid, an antiparasitic composition, or a combination thereof.

14. (canceled)

15. The composition according to claim 1 further comprising a pH adjusting agent, preservative, emulsifier, opacifier, antioxidant, fragrance, colorant, gelling agent, thickening agent, stabilizer, or surfactant.

16. The composition according to claim 4, wherein the antiviral agent is selected from the group consisting of abacavir, aciclovir, adefovir, amantadine, amprenavir, arbidol., atazanavir, artipla, brivudine, cidofovir, combivir, edoxudine, efavirenz, emtricitabine, enfuvirtide, entecavir, fomvirsen, fosamprenavir, foscarnet, fosfonet, ganciclovir, gardasil, ibacitabine, immunovir, idoxuridine, imiquimod, indinavir, inosine, integrase inhibitors, interferons, including interferon type III, interferon type II, interferon type I, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, tenofovir, tenofovir disoproxil, tipranavir, trifluridine, trizivir, tromantadine, truvada, valganciclovir, vicriviroc, vidarabine, viramidine, zalcitabine, zanamivir, zidovudine, and combinations thereof.

17. The composition according to claim 4, wherein the antibacterial agent is selected from the group consisting of amikacin, gentamicin, kanamycin, neomycin, netilmicin, streptomycin, tobramycin, paromomycin, geldanamycin, herbimycin, loracarbef, ertapenem, doripenem, imipenem, cilastatin, meropenem, cefadroxil, cefazolin, cefalotin, cefalexin, cefaclor, cefamandole, cefoxitin, defprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillan, bacitracin, colistin, polymyxin B, ciprofloxacin, enoxacin, gatifloxacin, levofloxacin, lomefloxacin, moxifloxacin, norfloxacin, ofloxacin, trovfloxacin, mafenide, prontosil, sulfacetamide, sulfamethizole, sulfanimilimde, sulfasalazine, sulfisoxazole, trimetoprim, demeclocycline, doxycycline, minocycline, oxytetracycline, tetracycline, arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinuspristin/dalfopristin, rifampin, tinidazole, AL-15469A, AL-38905, and combinations thereof.

18. The composition according to claim 4, wherein the antifungal agent is selected from the group consisting ofamrolfine, utenafine, naftifine, terbinafine, flucytosine, fluconazole, itraconazole, ketoconazole, posaconazole, ravuconazole, voriconazole, clotrimazole, econazole, miconazole, oxiconazole, sulconazole, terconazole, tioconazole, nikkomycin Z, caspofungin, micafungin, anidulafungin, amphotericin B, liposomal nystastin, pimaricin, griseofulvin, ciclopirox olamine, haloprogin, tolnaftate, undecylenate, clioquinol, and combinations thereof.

19. The composition according to claim 4, wherein the antiparasitic agent is selected from the group consisting ofamitraz, amoscanate, avermectin, carbadox, diethylcarbamizine, dimetridazole, diminazene, ivermectin, macrofilaricide, malathion, mitaban, oxamniquine, permethrin, praziquantel, prantel pamoate, selamectin, sodium stibogluconate, thiabendazole, and combinations thereof.

20. The composition according to claim 4, wherein the corticosteroid is selected from the group consisting ofhydrocortisone, prednisone, fluprednisolone, triamcinolone, dexamethasone, betamethasone, cortisone, prednilosone, methylprednisolone, fluocinolone acetonide, flurandrenolone acetonide, and fluorometholone.
